(19) United States
(12) Patent Application Publication
(10) Pub. No.: US 2010/0093995 A1
(43) Pub. Date: Apr. 15, 2010

(54) PROCESS FOR THE RECOVERY OF HCL FROM A DILUTE SOLUTION THEREOF

(75) Inventors: Avram Baniel, Jerusalem (IL); Aharon Eyal, Jerusalem (IL)

Correspondence Address: LUCAS & MERCANTI, LLP, 475 PARK AVENUE SOUTH, 15TH FLOOR, NEW YORK, NY, 10016, US

(73) Assignee: HCL CLEANTECH LTD., Tel Aviv (IL)

(21) Appl. No.: 12/530,505

(22) PCT Filed: Mar. 4, 2008
(86) PCT No.: PCT/IL08/00278

§ 371 Date: Sep. 9, 2009

(30) Foreign Application Priority Data
Mar. 15, 2007 (IL) 181944
Feb. 24, 2008 (IL) 198699

(51) Int. Cl. C08B 1/00; C01B 7/01

(52) U.S. Cl. 536/56; 423/488; 536/123.1

(57) ABSTRACT

The invention provides a process for the recovery of HCl from a dilute solution thereof, comprising: a) bringing a dilute aqueous HCl solution into contact with a substantially immiscible extractant, said extractant comprising: 1) an oil soluble amine which amine is substantially water insoluble both in free and in salt form; 2) an oil soluble organic acid which acid is substantially water insoluble both in free and in salt form; and 3) a solvent for the amine and organic acid; whereupon HCl selectively transfers to said extractant to form an HCl-carrying extractant; and b) treating said HCl-carrying extractant to obtain gaseous HCl.

The present invention relates to a process for the recovery of hydrochloric acid from a dilute solution thereof, as well as to a process for the production of carbohydrates from a polysaccharide by acid hydrolysis with concentrated hydrochloric acid.

The term “hydrochloric acid,” as used in the present specification, is intended to denote all forms of hydrochloric acid, including aqueous solutions of hydrogen chloride (HCl) and gaseous phases containing the same. Such acid solutions are broadly present in industrial practice. They are used as reagents (e.g., in regeneration of ion-exchangers) and are formed as by-products or co-products of other processes. In the latter case, the hydrochloric acid obtained is frequently quite dilute, typically 5% HCl to 10% HCl, and needs be reconcentrated to the range of over 20%—desirably to about 30%—to be of commercial viability. The alternative of neutralization and disposal is inherently costly.

Concentration of hydrochloric acid by distillation is a well-known technology practiced for many years. Its basic drawback is the high cost of the equipment and the inherent large energy consumption. If various impurities are present in the dilute hydrochloric acid, the concentration by distillation needs to be preceded by some separation step to prevent equipment fouling or contamination of the concentrated hydrochloric acid.

In U.S. Pat. No. 4,291,007 by the present inventor, there is described and claimed a solvent extraction process for the separation of a strong mineral acid from other species present in an aqueous solution and the recovery thereof under reversible conditions utilizing an extractant phase that contains an acid-base-couple (hereinafter referred to as an “ABC solvent”) which obviates the consumption of chemicals for regeneration, comprising the steps of:
a) bringing an aqueous solution containing the mineral acid to be separated into contact with a substantially immiscible extractant phase, said extractant phase comprising:
   1) a strong organic acid, which acid is oil-soluble and substantially water-immiscible, in both free and salt forms;
   2) an oil-soluble amine, which amine is substantially water-insoluble, in both free and salt forms; and
   3) a carrier solvent for said organic acid and said amine, wherein the molar ratio of said organic acid to said amine is between about 0.5:2 and 2:0.5,
   whereupon said predetermined mineral acid selectively and reversibly transfers to said extractant phase;
b) separating said two phases; and
c) backwashing said extractant phase with an aqueous system to recover substantially all the mineral acid contained in said extractant phase.

The strong organic acids envisioned for use in the extractant phase of said invention were organic acids which may be defined and characterized as follows: When 1 mol of the acid in a 0.2 molar or higher concentration is contacted with an equivalent amount of 1N NaCl, the pH of the sodium chloride solution decreases to below 3.

Especially preferred for use in said invention were strong organic acids selected from the group consisting of aliphatic and aromatic sulfonic acids and alpha-, beta- and gamma-chloro and bromo-substituted carboxylic acids, e.g., hexadecylsulfonic acid, didodecylnaphthalene disulfonic acid, alpha-bromo lauric acid, beta, beta-dichloro decanoic acid and gamma dibromo octanoic acid, etc.

The amines of said invention are preferably primary, secondary and tertiary amines singly or in mixtures and characterized by having at least 10, and preferably at least 14, carbon atoms and at least one hydrophobic group. Such commercially available amines as Primene JM-5, and Primene JM-T (which are primary aliphatic amines in which the nitrogen atom is bonded directly to a tertiary carbon atom) and which commercial amines are sold by Rohm and Haas chemical Co.; Amberlite LA-1 and Amberlite LA-2, which are secondary amines sold by Rohm and Haas; Alamine 336, a tertiary tricaprylyl amine (TCA) and Alamine 304, a tertiary trilaurylamine (TLA), both sold by General Mills, Inc., can be used in the processes of said invention, as well as other well-known and available amines, including, e.g., those secondary and tertiary amines listed in U.S. Pat. No. 3,458,282.

The carrier solvents can be chosen from a wide range of organic liquids known to persons skilled in the art which can serve as solvents for said acid-amine active components and which provide for greater ease in handling and extracting control. Said carrier solvents can be unsubstituted or substituted hydrocarbon solvents in which the organic acid and amine are known to be soluble and which are substantially water-insoluble, e.g., kerosene, mineral spirits, naphtha, benzene, xylene, toluene, nitrobenzene, carbon tetrachloride, chloroform, trichloroethylene, etc. Also higher oxygenated compounds such as alcohols, ketones, esters, ethers, etc., that may confer better homogeneity and fluidity and others that are not acids or amines, but which may confer an operationally useful characteristic, can also be included.

In the process of said invention, the essential operating extractant is believed to be the amine, balanced by a substantially equivalent amount of strong organic acid. An excess of acid acts as a modifier of the system, and so does an excess of amine, which obviously will be present as salts of acids present in the system. These modifiers are useful in optimization of the extractant, but are not essential.

Thus, as stated, the molar ratio between the two foregoing active constituents lies between 0.5 to 2 and 2 to 0.5, and preferably between about 0.5 to 1 and 1 to 0.5.

The process as exemplified in said patent was especially useful for use with acids such as nitric acid; however, the process as defined therein wherein the acid is recovered by backwashing is not practical or commercially viable for obtaining concentrated hydrochloric acid from dilute hydrochloric acid.

According to the present invention, it has now been surprisingly found that HCl can be distilled out of such an HCl-loaded extractant phase at temperatures below 250° C. without noticeable solvent decomposition.

Thus, according to the present invention there is now provided a process for the recovery of HCl from a dilute solution thereof, comprising:
a) bringing a dilute aqueous HCl solution into contact with a substantially immiscible extractant, said extractant comprising:
   1) an oil soluble amine, which amine is substantially water-insoluble, in both free and salt forms;
   2) an oil soluble organic acid, which acid is substantially water-insoluble, in both free and salt forms; and
   3) a solvent for the amine and organic acid;
      whereupon HCl selectively transfers to said extractant to form an HCl-carrying extractant; and
b) treating said HCl-carrying extractant to obtain gaseous HCl.

The term “dilute HCl solution,” as used herein, is intended to refer to an aqueous solution comprising HCl and optionally other solutes, wherein the water/HCl w/w ratio is greater than 3, e.g. greater than 4, 6, 8 and 10. In many cases, the concentration of HCl in the solution is sub-azeotropic.”

The terms “extractant” and “ABC extractant” are used herein interchangeably.

The organic acids envisioned for use in the extractant phase of the present invention are organic acids which may be defined and characterized as follows: When 1 mol of the acid in a 0.2 molar or higher concentration is contacted with an equivalent amount of 1N NaCl, the pH of the sodium chloride solution decreases to below 3.

Especially preferred for use in the present invention are organic acids selected from the group consisting of aliphatic and aromatic sulfonic acids and alpha-, beta- and gamma-chloro and bromo substituted carboxylic acids, e.g., hexadecylsulfonic acid, didodecylnaphthalene disulfonic acid, alpha-bromo lauric acid, beta-, beta-dichloro decanoic acid and gamma dibromo octanoic acid, etc. and organic acids with at least 6, preferably at least 8, and most preferably at least 10, carbon atoms.

The amines of the present invention are preferably primary, secondary and tertiary amines singly or in mixtures and characterized by having at least 10, preferably at least 14, carbon atoms and at least one hydrophobic group. Such commercially available amines as Primene JM-5, and Primene JM-T (which are primary aliphatic amines in which the nitrogen atom is bonded directly to a tertiary carbon atom) sold by Rohm and Haas Chemical Co.; Amberlite LA-1 and Amberlite LA-2, which are secondary amines sold by Rohm and Haas; Alamine 336, a tertiary tricaprylyl amine (TCA) and Alamine 304, a tertiary trilaurylamine (TLA), both sold by General Mills, Inc., can be used in the processes of the present invention, as well as other well known and available amines including, e.g., those secondary and tertiary amines listed in U.S. Pat. No. 3,458,282.

The term “solvent,” as used herein, is intended to refer to any water-immiscible organic liquid in which the acid and amine dissolve. Hydrocarbons, alkanols, esters, etc. having the required immiscibility can be used individually or in admixtures.

In preferred embodiments of the present invention, the solvent is a hydrocarbon.

To avoid any misunderstanding, it is to be noted that the term “solvent,” as used herein, relates to the third component of the extractant.

The term “pH half neutralization (pHhn),” as used herein refers to an aqueous solution, the pH of which is in equilibrium with the extractant carrying HCl at an HCl-to-amine molar/molar ratio of 1:2.

In preferred embodiments of the present invention, said process further comprises:
c) absorbing the gaseous HCl to provide hydrochloric acid of a higher concentration than that of the HCl in said dilute solution.

Preferably, said treating comprises heating.

The present invention further provides a process as described hereinabove wherein said heating is at a temperature of up to 250° C., preferably not exceeding 200° C.

In some preferred embodiments of the present invention, said treating comprises introducing a stream of an inert gas for conveying the HCl from said extractant phase.

In other preferred embodiments of the present invention, said treating comprises a combination of heating and introducing a stream of an inert gas.

In yet another preferred embodiment of the present invention, said inert gas is a superheated steam.

In another aspect of the present invention, there is provided a process for the production of carbohydrates, comprising:
a) providing a polysaccharide
b) hydrolyzing said polysaccharide in an HCl-containing hydrolysis medium to form a carbohydrate-containing, dilute aqueous HCl solution;
c) bringing said dilute aqueous HCl solution into contact with a substantially immiscible extractant, said extractant comprising:
   1) an oil-soluble amine, which amine is substantially water-insoluble, in both free and salt forms;
   2) an oil-soluble organic acid, which acid is substantially water-insoluble, in both free and salt forms; and
   3) a solvent for the amine and organic acid,
   whereupon HCl selectively transfers to said extractant to form an HCl-carrying extractant and an HCl-depleted hydrocarbon-containing solution;
d) treating said HCl-carrying extractant to obtain gaseous HCl; and
e) using said gaseous HCl for hydrolysis of a polysaccharide.

In this aspect of the present invention, said process preferably further comprises a step (f), wherein said gaseous HCl gas is directly absorbed into a slurry of a comminuted polysaccharide-containing material to generate said HCl-containing hydrolysis medium.

Preferably, said polysaccharide-containing material is a lignocellulosic material

In preferred embodiments of the present invention, said HCl-depleted carbohydrate-containing solution provides a feedstock for fermentation to generate a fermentation product.

Preferably, said fermentation product is ethanol.

In some preferred embodiments of the present invention, the amount of HCl in said gaseous HCl is at least 70% of the amount of HCl in said dilute aqueous HCl solution, preferably at least 80%, and most preferred, at least 90%.

Preferably, at least 70% of the polysaccharide in said comminuted polysaccharide-containing material is hydrolyzed to carbohydrates. In especially preferred embodiments of the present invention, at least 80% of the polysaccharide is hydrolyzed to carbohydrates, and most preferred, at least 90% of the polysaccharide is hydrolyzed to carbohydrates.

In preferred embodiments of the present invention, said carbohydrate concentration in said HCl-depleted carbohydrate-containing solution is at least 15%. In especially preferred embodiments of the present invention, said carbohydrate concentration in said HCl-depleted carbohydrate-containing solution is at least 20%, and in the most preferred embodiments of the present invention, it is at least 30%.

In some preferred embodiments of the present invention, said polysaccharide is provided in a polysaccharide-containing material, said process further comprising a step of comminuting said material to form a slurry, wherein said provided polysaccharide material has not been dried prior to said forming of said slurry.

While the invention will now be described in connection with certain preferred embodiments in the following examples and with reference to the appended figures so that aspects thereof may be more fully understood and appreciated, it is not intended to limit the invention to these particular embodiments. On the contrary, it is intended to cover all alternatives, modifications and equivalents as may be included within the scope of the invention as defined by the appended claims. Thus, the following examples which include preferred embodiments will serve to illustrate the practice of this invention, it being understood that the particulars shown are by way of example and for purposes of illustrative discussion of preferred embodiments of the present invention only and are presented in the cause of providing what is believed to be the most useful and readily understood description of formulation procedures as well as of the principles and conceptual aspects of the invention.

In the drawings:

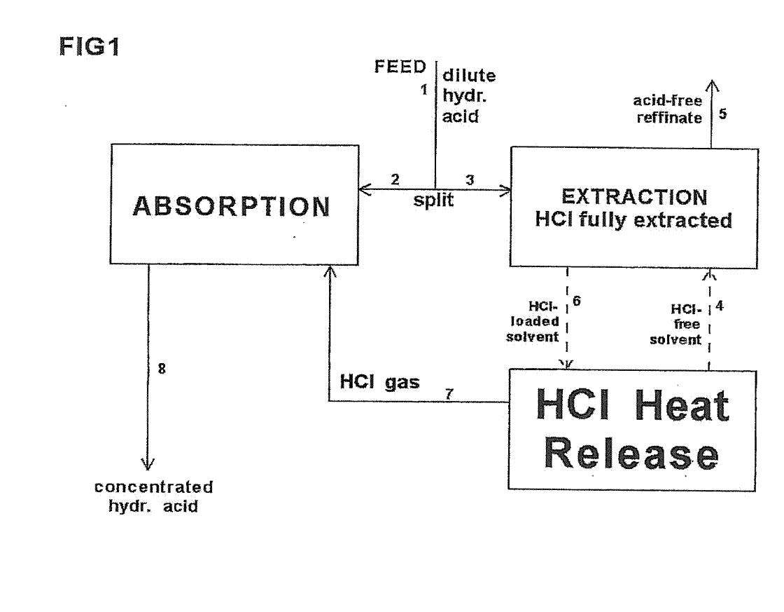

FIG. 1 is a schematic flow diagram of recovery of HCl only from a part of a feed;

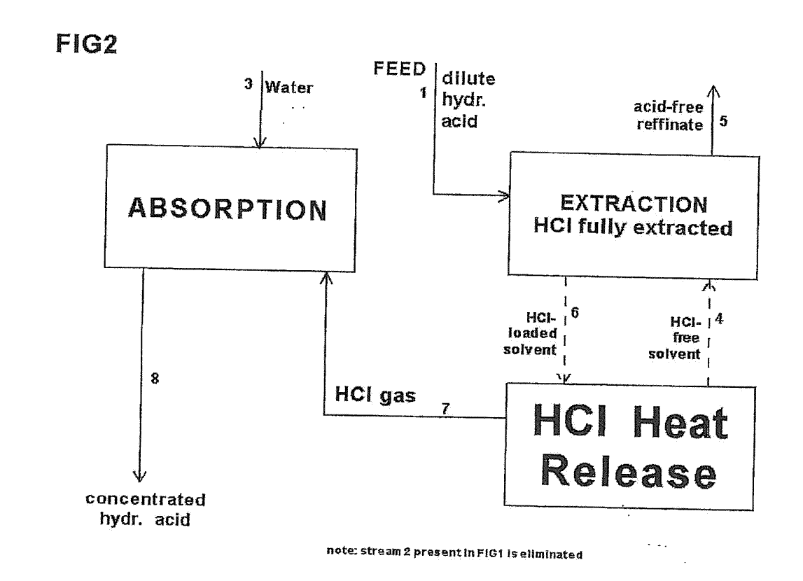

FIG. 2 is a schematic flow diagram of recovery of all of the HCl in the feed and absorption in water; and

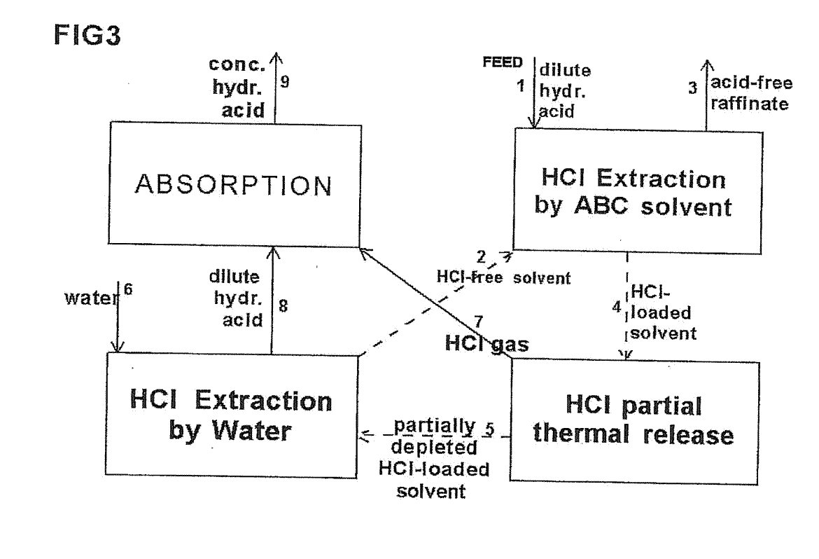

FIG. 3 is a flow diagram of release of HCl from the extractant phase partly thermally and partly by extraction via liquid-liquid-contacting.

### EXAMPLES

### Illustrative Example 1

A round-bottomed flask containing 20 ml of a simulated HCl-loaded extract was placed in an oil bath maintained at 180° C. The simulated extract consisted of a solution in mineral oil (boiling point above 250° C.) containing 0.2 meq/ml dinonylnaphthalene sulfonic acid (HDNNS) and 0.2 meq/ml tridodecylamine hydrochloride (C₁₂H₂₅)₃N.HCl. A stream of nitrogen gas of about 2 ml/min was passed through the organic extract and exited through a water trap. After 90 minutes the nitrogen was stopped and the HCl in the water trap titrated. In two replications of the experiment, 98.5% and 99.3% of the HCl in the organic solution were recovered. In each experiment the remaining mineral oil was contacted with aqueous 5% NaOH and the aqueous phases checked for Cl⁻. Hardly any CI could be perceived.

### Illustrative Example 2

A 40 ml solution of the same solutes as above, 0.1 meq of each, was prepared in a non-aromatic petroleum extract (described as 98% boiling at 172° C./195° C.) simulating an HCl-carrying extract. The organic liquid was placed in a flask that was heated in a controlled fashion to distill the contents slowly, without reflux, directly into a cooled water trap. The distillation was stopped in 55 minutes when about 20 ml distillate was collected. All of the HCl in the simulated extract was found in the aqueous phase in the trap. None could be determined in the approximately 20 ml liquid that remained in the flask.

These examples demonstrate that HCl carried by ABC extractants can be recovered as HCl gas by heating to a temperature that needs not exceed 200° C. while providing an inert carrier for conveying the HCl. Taken with the known art of extracting HCl from aqueous solutions thereof (as provided in U.S. Pat. No. 4,291,007 cited above) provides for designing a great variety of schemes for concentrating hydrochloric acid. For each practical problem, a practitioner can resort to the large choice of ABC extractants and the particular demands of each case.

Three general cases are represented by FIGS. 1,2 and 3, and are discussed with reference to these figures.

The case schematized in FIG. 1 recovers only the HCl from a part (3) of feed (1) and the HCl gas thus recovered (7) is absorbed in part (2) of feed (1), to obtain a concentrated hydrochloric acid (8). Thus, for example, a feed (1) of 10.71% HCl (12 HCl per 100H₂O) split equally between (2) and (3) will provide a product (8) of 19.4% HCl; if split in a ratio of (3):(2)=2:1, the product (8) will have a concentration of 26.4% HCl.

The case schematized in FIG. 2 recovers all of the HCl in feed (1) and absorbs it in water, which provides for easy control of concentration and for purity of the product HCl solution (8).

A useful variant of this general procedure is to absorb the HCl gas directly in an aqueous medium of a process that requires concentrated hydrochloric acid, for instance, a slurry of a comminuted cellulosic material due to be hydrolyzed.

The release of HCl from an ABC extractant extract can be divided into two parts: thermal—which recovers HCl partially as gas, and liquid-liquid extraction by water—which recovers the remainder of the HCl as dilute hydrochloric acid that absorbs the HCl gas thermally released—as schematized in FIG. 3.

These are just three of the numerous flow sheet varieties, each of which can be conceived and elaborated in detail to fit the particulars of each case that involves hydrochloric acid concentration. One example is detailed below by way of illustration.

### Illustrative Example 3

The scheme shown in FIG. 2 was used in laboratory simulation of HCl recycle for an industrial process related to cellulose conversion to glucose by acid hydrolysis. In this process a 32% acid is used to effect the hydrolysis. The HCl (which acts as catalyst and is not consumed) reports to a clarified aqueous product solution containing 172 grs/L HCl (4.7 molar and about 22% HCl with respect to the water in this product) that need be recovered as hydrochloric acid of 32%.

The HCl extraction was run in a battery of six laboratory mixer-settlers. The solvent was 0.52 molar in an ABC of 1:1 TLA:HDNNS (trilaurylamine-dinonylnaphtalene sulfonic acid) in a hydrocarbons diluent of a boiling range starting at 210° C. The volumetric ratio of extractant (stream 4 in FIG. 2) to aqueous feed (stream 1 in FIG. 2) was 10:1. The pH of the aqueous raffinate stabilized at 6.2, indicating that the extraction of HCl was practically complete. The solvent extract (stream 6 in FIG. 2) was 0.46 molar in HCl.

100 ml of this extract were heated in a glass vessel to 160° C. by immersion in a thermostatic bath maintained at this temperature. Steam superheated to 160° C./170° C. (generated by passing water through a heated copper pipe) was sparged into the liquid extract to serve as carrier for the HCl released. The gaseous mixture of H₂O and HCl was passed through an externally refrigerated graphite pipe, whereby condensation to hydrochloric acid took place. The experiment was repeated with varying amounts of steam, with each replicated to obtain safe averages.

The tabulated results are as follows:

| Condensate, gms | % HCl | Extent of release |
|---|---|---|
| 4.7 | 33.1 | Incomplete |
| 5.2 | 32.4 | Nearly complete |
| 5.5 | 30.6 | Complete |
| 5.7 | 29.6 | Complete |
| 6.0 | 28.1 | Complete |

These results clearly indicate that recovery of the HCl at the higher concentration required for cellulose hydrolysis is feasible.

Cellulose hydrolysis by hydrochloric acid is very efficient and provides a hydrolysate of desirable properties. However, the high costs of hydrochloric acid re-concentration made it inapplicable.

The present invention provides a solution to this problem, as described and exemplified hereinabove, by providing an economical process for recycling and reconcentration of hydrochloric acid.

It will be evident to those skilled in the art that the invention is not limited to the details of the foregoing illustrative examples and that the present invention may be embodied in other specific forms without departing from the essential attributes thereof, and it is therefore desired that the present embodiments and examples be considered in all respects as illustrative and not restrictive, reference being made to the appended claims, rather than to the foregoing description, and all changes which come within the meaning and range of equivalency of the claims are therefore intended to be embraced therein.

What is claimed is:

1. A process for the recovery of gaseous HCl from a dilute solution thereof, comprising:
a) bringing a dilute aqueous HCl solution Into contact with a substantially Immiscible extractant, said extractant comprising;
1) an oil soluble amine, which amine is substantially water Insoluble both in free and in salt form;
2) an oil soluble organic acid, which acid is substantially water Insoluble both in free and In salt form; and
3) a solvent for the amine and organic acid;
whereupon HCl selectively transfers to said extractant to form an HCl-carrying extractant; and
b) distilling MCI from said HCl-carrying extractant to form gaseous HCl and HCl-depleted extractant.

2. A process according to claim 1, further comprising
c) absorbing the gaseous HCl produced in step b In a part of said dilute solution or in water, to provide hydrochloric acid of a higher concentration than that of the HCl In said dilute aqueous HCl solution of step a.

3. A process according to claim 1, wherein said distilling comprises heating.

4. A process according to claim 3, wherein said heating is to a temperature of up to 260° C.

5. A process according to claim 4, wherein said heating is to a temperature of up to 200° C.

6. A process according to claim 1, wherein said distilling comprises introducing a stream of an Inert gas for conveying the MCI from said extraotant phase.

7. A process according to claim 1, wherein said distilling comprises a combination of heating and Introducing a stream of an inert gas.

8. A process according to claim 6, wherein said inert gas is a superheated steam.

9. A process according to claim 1, wherein said extractant is characterized by a pHhn of less than 3.

10. A process for the production of carbohydrates, comprising:
a) providing a polysaccharide
b) hydrolyzing said polysaccharide in an HCl-containing hydrolysis medium to form a carbohydrate-containing, dilute aqueous HCl solution;
a) bringing said dilute aqueous HCl solution Into contact with a substantially immiscible extractant, said extractant comprising;
1) an oil-soluble amine, which amine is substantially water-insoluble, in both free and salt forms;
2) an oil-soluble organic acid, which acid is substantially water-insoluble, in both free and salt forms; and
3) a solvent for the amine and organic acid,
whereupon HCl selectively transfers to said extractant to form an HCl-carrying extractant and an HCl-depleted carbohydrate-containing solution;
d) distilling HCl from said HCl-carrying extractant to form gaseous 1101 and HCl-depleted extractant and
e) using said gaseous HCl for hydrolysis of a polysaccharide.

11. A process according to claim 10 wherein said polysaccharide is provided in a polysaccharide-containing material.

12. A process according to claim 11 further comprising a step of comminuting said material to form an aqueous slurry.

13. A process according to claim 11, wherein said polysaccharide-containing material is a lignocellulosic material

14. A process according to claim 10, wherein said HCl-depleted carbohydrate-containing solution provides a feedstock for fermentation to generate a fermentation product.

15. A process according to claim 10, wherein said fermentation product is ethanol.

16. A process) according to claim 10, wherein the amount of HCl in said gaseous HCl is at least 70% of the amount of HCl in said dilute aqueous HCl solution.

17. A process according to claim 11, wherein at least 70% of the polysaccharide in said polysaccharide-containing material is hydrolyzed to carbohydrates,

18. A process according to claim 10, wherein carbohydrate concentration in said HCl-depleted carbohydrate-containing solution is at least 15%.

19. A process according to claim 10, wherein said polysaccharide is provided in a polysaccharide-containing material, said process further comprising a step of comminuting said material to form a slurry, wherein said provided polysaccharide material has not been dried prior to said forming of said slurry.

20. A process according to claim 19, further comprising a step wherein said gaseous HCl is directly absorbed into a slurry of a comminuted polysaccharide-containing material to generate said HCl-containing hydrolysis medium.

21. A process according to claim 10, wherein said extractant is characterized by a pHhn of less than 3.
